(19) United States
(12) Patent Application Publication
(10) Pub. No.: US 2008/0188663 A1
(43) Pub. Date: Aug. 7, 2008

(54) Process for the preparation of crystalline clopidogrel hydrogen sulphate Form I

(76) Inventors: Ashok Kumar, Mumbai (IN); Priti J. Bhayani, Mumbai (IN); Vaibhav Chinubhai Doshi, Mumbai (IN); Ashvini Saxena, Pin (IN); Gunjan Pramod Pathak, Pin (IN); Rashmi Abhyankar, Pin (IN); Sarvanan Manavalan, Pin (IN); Mukesh Purohit, Pin (IN)

Correspondence Address: BLANK ROME LLP, 600 NEW HAMPSHIRE AVENUE, N.W., WASHINGTON, DC, 20037, US

(21) Appl. No.: 12/010,581

(22) Filed: Jan. 28, 2008

(30) Foreign Application Priority Data
Jan. 29, 2007 (IN) 161/MUM/2007
Feb. 13, 2007 (IN) 292/MUM/2007
Aug. 20, 2007 (IN) 1594/MUM/2007

(51) Int. Cl. C07D 221/04

(52) U.S. Cl. 546/114

(57) ABSTRACT

The present invention describes an improved industrial process for crystallizing polymorph Form I of (+) clopidogrel hydrogen sulphate (also called clopidogrel bisulphate) in either a Type-I solvent or a Type-II. Type I solvent is an organic solvent containing two or more functional groups with a proviso that at least one functional group is different from the other(s). The Type II solvent is selected from methyl ethyl ketone, cyclopentylmethyl ether, dipropylglycolether, dibutylglycol ether, propylmethyl cellosolve, butylmethylcellosolve, propylethylellosolve, butylethylcellosolve or their cross combinations. The process produces Form I in a reproducible manner without detectable contamination of polymorph designated as Form II.

This patent application claims priority from our Indian patent applications: 161/MUM2007, filed on Jan. 29, 2007; 292/MUM/2007, filed on Feb. 13, 2007; and 1594/MUM/2007, filed on Aug. 20, 2007; the contents of which are incorporated herein by reference.

### FIELD OF INVENTION

The present invention relates to a process for preparing (+)-(S)-alpha-2-(chlorophenyl)-6,7-dihydrothieno[3,2-C]pyridine-5-(4-H)-acetic acid methyl ester hydrogen sulphate of Formula I, commonly known as clopidogrel bisulphate, in “Form-I” crystalline form.

### BACKGROUND OF THE INVENTION

(+)-(S)-alpha-2-(chlorophenyl)-6,7-dihydrothieno-[3,2-C]-pyridine-5-(4-H)-acetic acid methyl ester known as clopidogrel under the International Non-Proprietary Name is marketed as a hydrogen sulphate salt. Clopidogrel is known for its platelet aggregating and anti-thrombotic properties and finds medicinal applications in this field. It can be represented by Formula-I, and was disclosed in U.S. Pat. No. 4,529,596 ('596 patent) in its racemic form for the first time.

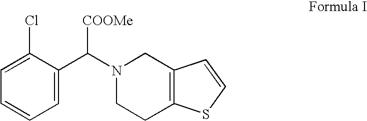

The pure enantiomeric forms of clopidogrel (dextro and levo isomers) were disclosed in EP281459 ('459 patent) which teaches the isolation of the dextro rotatory isomer of clopidogrel by diasteriomeric salt formation of racemic clopidogrel base using an optically active acid, such as L-camphor-10-sulfonic acid. The clopidogrel free base was then converted into its hydrogen sulfate salt by dissolving in acetone, cooling and mixing with concentrated sulfuric acid to precipitation. The precipitate thus obtained is then isolated by filtration, washed and dried to give clopidogrel hydrogen sulfate in the form of white crystals whose melting point was 184° C. and optical rotation was +55.1° (c=1.891/CH3OH). But '459 patent did not characterize or suggest any name to this crystals (polymorph) of clopidogrel hydrogen sulfate.

Subsequently WO 99/65915 ('915 application), disclosed two polymorphic forms of clopidogrel hydrogen sulfate referred to as Form I and Form II. The '915 patent identified that the precipitation method described in '459 patent had led to crystalline Form-I. The '915 also deals with a new crystalline form called Form-II of clopidogrel hydrogen sulfate. This Form II is suggested to be thermodynamically most stable crystalline form. According to '915 patent both polymorphs, namely Form I and Form II, were prepared from the same solvent viz; acetone.

The process for obtaining crystalline Form I of clopidogrel hydrogen sulfate according to example 1A of the '915 patent describes the introduction of clopidogrel camphor sulfonate in methylene dichloride (MDC) and transformation of this salt into clopidogrel base with potassium carbonate solution. Clopidogrel base is extracted in MDC; and the solvent is evaporated. Residue obtained is then dissolved in acetone and cooled. Addition of sulfuric acid is said to precipitate out clopidogrel hydrogen sulfate in Form I. Also in the same application, it was described to obtain Form II either by keeping the mother liquor of Form I for prolonged periods or by heating the acetone solution containing the base after addition of sulfuric acid to reflux or by subjecting the suspension to mechanical shearing using a shearing device or by inoculation.

However, this process was not found to be suitable for the production of Form I of clopidogrel hydrogen sulphate on an industrial scale owing to its thermodynamic instability in solvents like acetone and invariably yielded Form II without having the need of keeping for longer periods (ref. '915 patent). This problem became the subject of many latter patent applications and a detailed study of the various publications clearly indicating that the manufacture of Form I of clopidogrel hydrogen sulphate poses a well known technical challenge to process chemists. Although most of the general class of organic solvents are said to be used for preparation of Form I polymorph, it appears both from the latest literature and from the experimental studies on this polymorph by the present inventors, most of these solvents gives only Form II or a Form I contaminated with Form II. The presence of Form II in Form I can lead to instability of Form I which results in inconsistency in formulations and ultimately leading to varying drug bioavailability. Moreover, among all these, most processes are not reliably reproducible on large scale.

The present inventors have also noted that, since the Form I is thermodynamically unstable, the process variants of dissolving clopidogrel hydrogen sulphate salt in conventional solvents at higher temperature and cooling to precipitate Form I resulted in Form II or its mixture with Form I. Moreover, the poor solubility of clopidogrel salt (whereas the free base possess good solubility) in most of the known solvents does not allow to use this crystallization process variant to be practiced.

There are ample literatures available for preparation of Form I, such as US2006074242, US20060205766, WO2005117866, WO2005100364, US2005059696, WO2005063708, WO2005016931, WO2005003139, SK12852002, PL355514, WO2004026879, WO2004081015, WO2004020443, WO2004081016, WO2004048385, WO2004081016, WO20040024012, WO2006087226, US20030114479, US2003225129, & WO2002059128 name a few, but it is also clear from these literature that same solvent unarguably gives two different crystalline forms in different hands, which is surprising. For example, US2003/225129 ('129 application) describes process for the preparation of Form-II from solvents selected from dichloromethane, 1,4-dioxane, toluene, chloroform, ethyl acetate, methyl ethyl ketone and t-butyl methyl ether. The '129 patent, for the first time, claimed to produce Form II from ethyl acetate which was the main subject of WO2004020443 for Form I preparation. A cursory review of the prior art indicates that almost all classes of solvents covering ethers, diethers, esters, alcohols, nitriles, ketones etc. are disclosed for use in the preparation of Form I, but those solvents are also taught to give Form II in the same or different publication.

WO2005003139 discusses combination of polar and non-polar solvent combinations for obtaining Form I, however, the present inventors have found that the adjustment of specific proportion of two or more solvents are rather difficult and does not give consistent and reproducible results while adjusting the polarity of mixtures.

So, it is evident from the prior art that the methods to produce Form-I of clopidogrel hydrogen sulphate from known solvents are poorly reproducible, necessitating the optimization of experimental conditions and/or the selection of suitable solvents. Since Form-I is kinetically controlled and Form-II is thermodynamically controlled form, they require very specific temperature range and specific conditions for getting reproducible results in conventional solvents. Also, in these solvents a minor variation in condition appears to give Form-II instead of expected Form-I or a mixture of Form-I & Form-II. Since, Form I of clopidogrel hydrogen sulphate is used for pharmaceutical formulation, the importance of a reliable method that gives Form I consistently is highly desirable.

Thus, there is a need for an industrially reliable process for the preparation of Form I crystals of clopidogrel hydrogen sulfate without contamination of Form II or other polymorphs.

### SUMMARY OF THE INVENTION

An objective of the present invention is to provide solvent systems where the Form I crystals of clopidogrel hydrogen sulphate can be efficiently and reproducibly formed and easily operated in large scale operations.

Another objective of the present invention is to provide a method for reliably making crystalline clopidogrel hydrogen sulfate in Form I, especially in large scale operation.

Yet another objective of the present invention is to provide crystalline clopidogrel hydrogen sulfate in Form I substantially free of other polymorphs, such as Form II.

The present inventors had identified that the prior art processes present substantial difficulties in producing Form I polymorph of clopidogrel hydrogen sulphate (also referred as clopidogrel bisulphate) in a consistent manner. The invention, therefore, aims to provide solvent(s) or solvent systems useful for making Form I clopidogrel hydrogen sulphate (Formula IB) in a consistent manner and processes for the same.

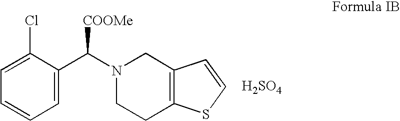

The present invention provides an improved industrial process for making polymorph Form I of (+) clopidogrel hydrogen sulphate (also called clopidogrel hydrogen sulphate) from a Type I or a Type II organic solvent, in a reproducible manner without detectable contamination of form II. Type I organic solvent is an organic solvent containing two or more functional groups with a proviso that at least one functional group is different from the other(s). A functional group, as used herein, refers to an atom or group of atoms that contains at least one atom that is not carbon and hydrogen. The functional groups may be halo functional groups, such as fluoro, chloro, bromo, iodo groups; oxygen containing functional groups, such as haloformyl, hydroxyl, carbonyl, aldehyde, ester, carboxyl, carboxylate, ether, and peroxy groups; nitrogen containing functional groups, such as amide, amine, imine, imide, azide, azo, cyanate, nitrate, nitrile, nitrite, nitro, nitroso, and pyridyl groups; phosphorus containing functional groups, such as phosphino, phosphate, and phosphono groups; and sulfur containing functional groups, such as sulfonyl, sulfo, sulfinyl, sulfhydryl, thiocyanate, and disulfide groups. Examples of multiple functional groups which can be present in the Type I solvent are selected from carbonyl-ester (ketone-ester), ether-carbonyl (ether-ketone), nitrile-ester, hydroxyl-ester (alcohol-ester), hydroxyl-ether (alcohol-ether), carbonyl-ether (ketone-ether), halogen substituted ester, nitro-ester, carbonyl-nitrile (ketone-nitrile), etc. Especially preferred Type I solvent possess carbon atoms ranging from C4 to C12 atoms. Specific examples of such solvents are ethyl acetoacetate, methyl acetoacetate, chloropropionyl acetate, alkyl lactates, chloroethylacetoacetate, chloroacetylacetoacetate etc. Type II solvent is selected from methyl ethyl ketone, cyclopentylmethyl ether, dipropylglycolether, dibutylglycol ether, propylmethyl cellosolve, butylmethylcellosolve, propylethylellosolve, butylethylcellosolve or their mixtures.

In a preferred embodiment of the present invention, the process for preparation of Form I crystals of clopidogrel hydrogen sulfate comprises dissolving the clopidogrel base in either Type I or Type II solvent as defined above; cooling the obtained clopidogrel base solution to a temperature of −20 to 30° C.; adding concentrated sulphuric acid (98%, d=1.84) while maintaining the temperature in the above range to obtain clopidogrel hydrogen sulphate salt; optionally further maintaining the salt at a temperature of about 10 to 30° C. and filtering the crystals of Form I obtained.

In a second embodiment of the present invention, the process for preparation of Form I comprises dissolving the clopidogrel hydrogen sulphate of any crystal form, for example, amorphous, Form II, or Form III, or their mixtures or contaminated Form I of clopidogrel hydrogen sulphate in a suitable Type I solvent as defined above; then cooling the obtained clopidogrel hydrogen sulphate solution to a temperature of −20 to 30° C. and maintaining the salt at a temperature of about 10 to 30° C. to complete precipitation of Form I and filtering the crystals of Form I obtained.

### BRIEF DESCRIPTION OF FIGURES

FIG. 1 represents Powder X-Ray diffraction pattern (PXRD) of clopidogrel hydrogen sulphate Form I prepared according to example 1 of the present invention.

FIG. 2 represents Differential Scanning Calorimetry record of Form I of clopidogrel hydrogen sulphate prepared according to example 1 of the present invention.

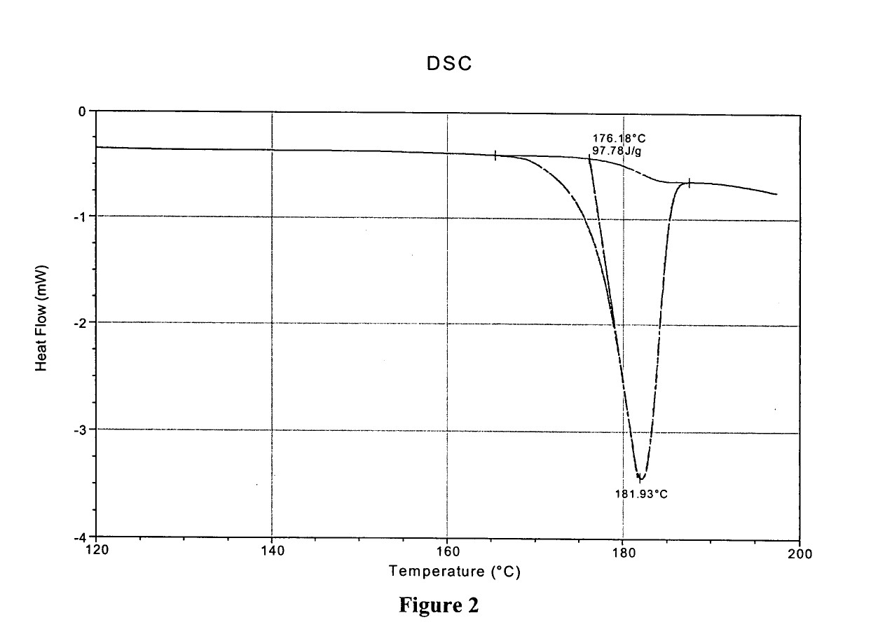

FIG. 3 represents Powder X-Ray diffraction pattern (PXRD) of clopidogrel hydrogen sulphate Form I standard as given in '915 patent.

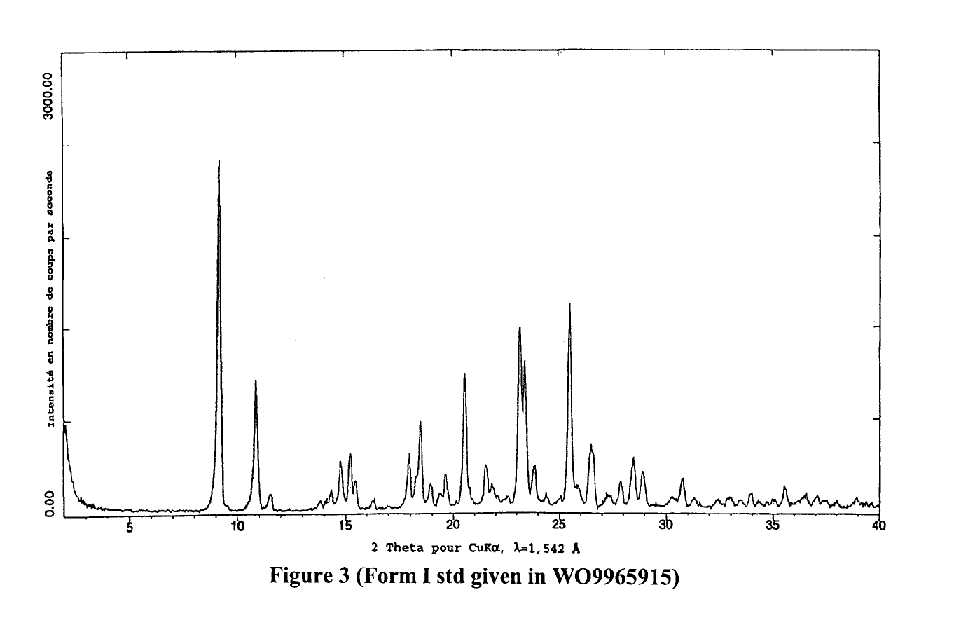

FIG. 4 represents the spectrogram obtained by Fourier Transform Infra Red spectrometry (FTIR) of clopidogrel hydrogen sulphate Form I prepared according to example 1 of the present invention.

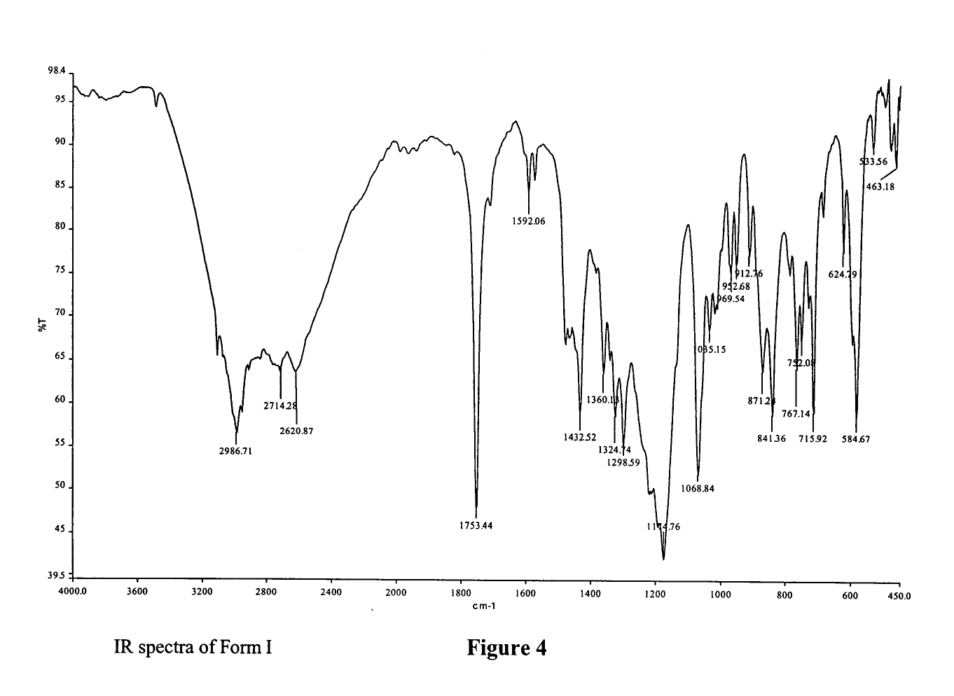

FIG. 5 represents an overlay of powder x-ray diffraction pattern (PXRD) of clopidogrel hydrogen sulphate crystalline Form I and Form II.

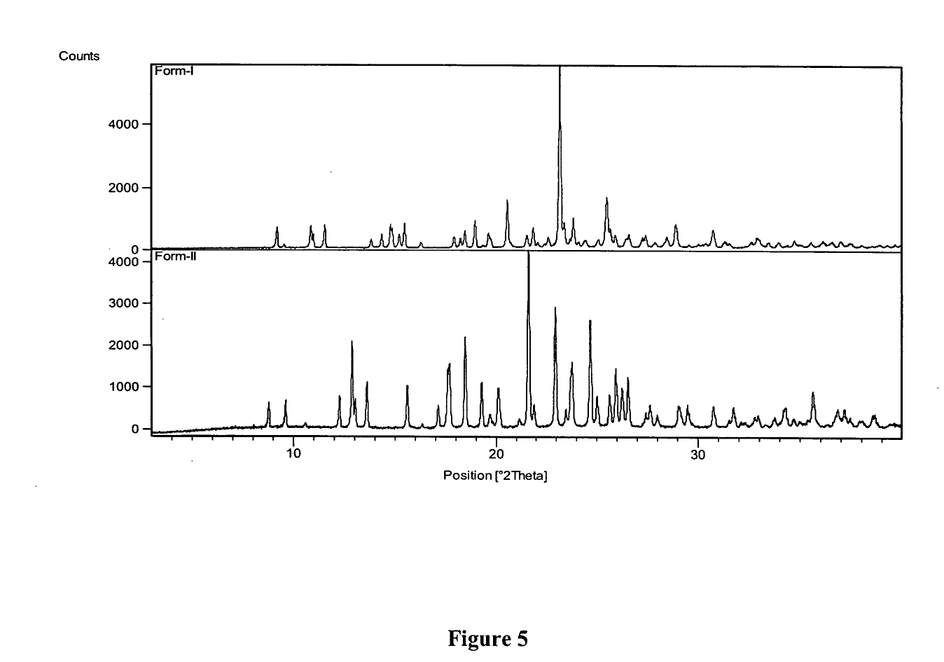

### DETAILED DESCRIPTION OF THE INVENTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The term “isolating” is used to indicate separation or collection or recovery of the compound being isolated in the specified crystalline form.

The term “separating from a solvent” with respect to the crystalline solids described herein means obtaining a solid of specified characteristics from a solution or a partial solution.

The term “treating” means adding or combining or mixing the stated reagent or materials to the things being treated.

As used herein the term “(+)-clopidogrel” or “(+)-(S)-clopidogrel” means the dextro-rotatory isomer of clopidogrel.

The term “forming a solution” means obtaining a solution of a substance in a solvent in any manner.

The term “inoculating” has the same meaning as the term “seeding,” and means adding previously obtained solid to facilitate crystallization. Thus, the term “seeding crystals” with respect to claimed process means crystals/powder of previously obtained crystalline Form I of clopidogrel hydrogen sulphate.

It should be understood that there exists equilibrium between a free species and salt form of a compound capable of forming salt with bases/acids (e.g., by virtue of having an amino-functionality in the molecule).

For the purposes of this description and claims of the present invention, the term “crystalline Form I” of clopidogrel hydrogen sulphate is the polymorphic form denoted as Form I and having characteristics as given in U.S. Pat. No. 6,429,210 patent. Identification of solids obtained by the process of the invention can be made by comparing with the reference analytical information provided in the U.S. Pat. No. 6,429,210 patent. Of course, it should be understood that operator, instrument and other similar variables may result in some margin of error with respect to analytical characterization of the solid.

Large scale production (greater than 40 g) of Form I according to the process described in the '915 patent or '210 patent resulted in Form II or a mixture with Form I or contaminated with other forms at a higher percentage. This lead us to find suitable solvents or conditions where these problems are minimized and ensure reproducibility of Form I, without contamination of other forms, especially Form II. This research has led to an efficient process where Form I can be produced reliably from a single solvent or mixture of solvents.

Thus the present inventors, on exploring various process alternatives, for a reliable process solution have found that the use of selected organic solvents which are categorized into either Type I or Type II solvents or the mixtures of such solvents for the crystallization of clopidogrel to reliably prepare Form I of clopidogrel hydrogen sulfate. Type I organic solvent possesses two or more functional groups, wherein at least one such functional group is distinctly different from other. The preferred functional groups in the Type-I solvent are either ester-ketone, or ether-ketone, or hydroxyl-esters, keto-esters, or ester-nitrile, or ether-ester, or nitro-esters etc. The preferred Type I solvent has carbon atoms from C4 to C12 in the molecule. The preferred Type I solvents are ethyl acetoacetate, methyl acetatoacetate, alkyl lactate, chloropropylacetate esters. 4-chloroethylacetoacetate esters, and chloroacetyl acetoacetate esters. Type-II solvent is a solvent selected from methyl ethyl ketone, cyclopentylmethyl ether, dipropylglycolether, dibutylglycol ether, propylmethyl cellosolve, butylmethylcellosolve, propylethylellosolve, butylethylcellosolve and their mixtures.

The Type I or Type II solvent may also be mixed with one or more other solvents. It has also been observed that presence of a specified concentration range of the other solvent(s) in the novel solvent(s) of the present invention does not alter results of the present process. Thus, a solvent selected from the group of esters, C3-C6 aliphatic or alicylcic ketones, C3-C6 chain or branched chain alcohols, and ethers in about 1 to 25 weight percent in a mixture with the Type I or Type II solvent of the present invention gives Form I consistently. The weight percent will depend upon the choice of the individual solvent from the above class of solvents, but can be determined by routine experimentation. Especially preferred other solvents for forming mixtures with the Type I or Type II solvent are ketones (such as acetone, methylpropyl ketone, methylisopropyl ketone, and isobutyl ketone), esters (such as propylacetate and butyl acetate), alcohols (such as butanol and pentanol among), and ethers (such as tertiary butyl methyl ether and cyclopentyl methyl ether). Most of these other solvents are not suitable independently for preparing pure Form I. Sometimes, the presence of these solvents in the process of the present invention are especially useful in removing the Type I solvent used in the invention to keep their level in the final product as low as possible to meet the pharmacopoeias specifications. It is very surprising to note that the reactive compounds like ethyl acetoacetate is inert through out the crystallization process of the present invention and does not participate in any significant chemical change or impurity formation as against the expected reactivity of such solvents.

Accordingly, in one embodiment, the present invention provides a process for preparing polymorph Form I of clopidogrel hydrogen sulphate comprising dissolving the (+)-clopidogrel base in either a Type-I or Type-II organic solvent or their mixtures, as defined/described before; cooling the obtained solution, preferably to a temperature of −25 to 30° C.; adding concentrated sulphuric acid (98%, d=1.84) while keeping the temperature in the range of −20 to 20° C.; and maintaining for a period of 5 to 10 hours at −20 to 30° C. to crystallize out the Form I clopidogrel hydrogen sulphate. The precipitated crystals may be collected by conventional methods, such as filtration or centrifugation. Although temperature lower than −20° C. also works while addition of sulfuric acid, owing to the industrial applicability the above range is preferred.

A mixture of Type-I or Type-II solvents can be mixed with each other and may be employed in all proportions.

In the process for preparation of Form I, the preferred concentration of sulphuric acid is in the range of 80% to 98% and the molar ratio of the sulfuric acid to the (+)-clopidogrel base is in the range of about 1:1 to about 1.1:1. The most preferred concentration of sulphuric acid used in the salt formation is 90-98%. The sulphuric acid may be employed directly in the salt formation or may be employed as a solution in a carrier solvent. Especially preferred carrier solvents, although not limited to, are those selected from the above described Type-I or Type-II group solvents. In a preferred embodiment of the process, the exotherm of sulphuric acid addition is controlled by cooling and maintaining the temperature in between −10 to 10° C.

The Form I so obtained was characterized by PXRD, DSC and FTIR without any detectable quantity of Form II or other polymorphic Forms with respect to the standard PXRD pattern of Form I as described in '915 patent. The Form-I obtained by the process of the present invention does not contain any detectable Form-II polymorph and therefore it is stable to storage/handling.

In the process, the clopidogrel free base solution may be obtained by dissolving clopidogrel free base in the solvent at any temperature at or below the reflux temperature of the solvent and the solution may then be filtered to remove any particulate matter. Once the solution of the clopidogrel free base is obtained, the solution is cooled to a temperature below 25° C., more particularly below 10° C., and sulfuric acid solution is incorporated to form the clopidogrel hydrogen sulphate salt. The mass is then cooled or maintained at this temperature until crystallization of the solid is complete. The solid is filtered, washed, and dried. In the process, optionally, either before or after sulphuric acid addition, the solution may be seeded (inoculated with seed crystals) with previously obtained crystals of the Form I. The seed crystals may be obtained by performing the present invention at a lower scale or by methods known in the art.

In a second embodiment of the present invention, a process for preparation of Form I is provided which comprises dissolving the clopidogrel hydrogen sulphate salt of any crystal form (for example, amorphous, Form II, or Form III, or their mixtures or contaminated Form I) in a Type-I organic solvent (for example, ethyl acetoacetate or alkyl lactate, or chloroethylacetoacetate); then cooling the obtained clopidogrel hydrogen sulphate solution to a first temperature, preferably −20 to 30° C.; maintaining the salt at a second temperature, preferably about 10 to 30° C. to complete precipitation of Form I; and filtering the crystals of Form I obtained. The dissolution of the clopidogrel salt in the solvent is carried out at a temperature from ambient to reflux temperature of the solvent, preferably from 50 to reflux temperature of the solvent. The solution is then allowed to cool to room temperature, optionally kept for a holding time and then cooled to −20 to 30° C., preferably 0 to 5° C. to precipitate the Form I clopidogrel hydrogen sulphate.

The starting material, clopidogrel free base may be obtained by following any known process disclosed in the literature. The present inventors used samples obtained as per the process disclosed in EP1723141. Analytical characterization of the solid(s) obtained in accordance with the process of the invention was carried out by using X-ray powder diffraction using a PANALYTICAL XpertPRO X-Ray machine of Philips make. The X-ray powder diffraction patterns were recorded with Cu K alpha-1 radiation source (voltage of 50 kV; current: 25 mA).

The stable clopidogrel bisulphate Form I obtained by the process of the present invention may be formulated into a dosage form, e.g., tablet, capsule, etc., by combining with one or more pharmaceutically acceptable excipients using known techniques. The resulting dosage form may include a suitable amount of the active ingredient required for the desired action. Further, the dosage form may be immediate release or extended release.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

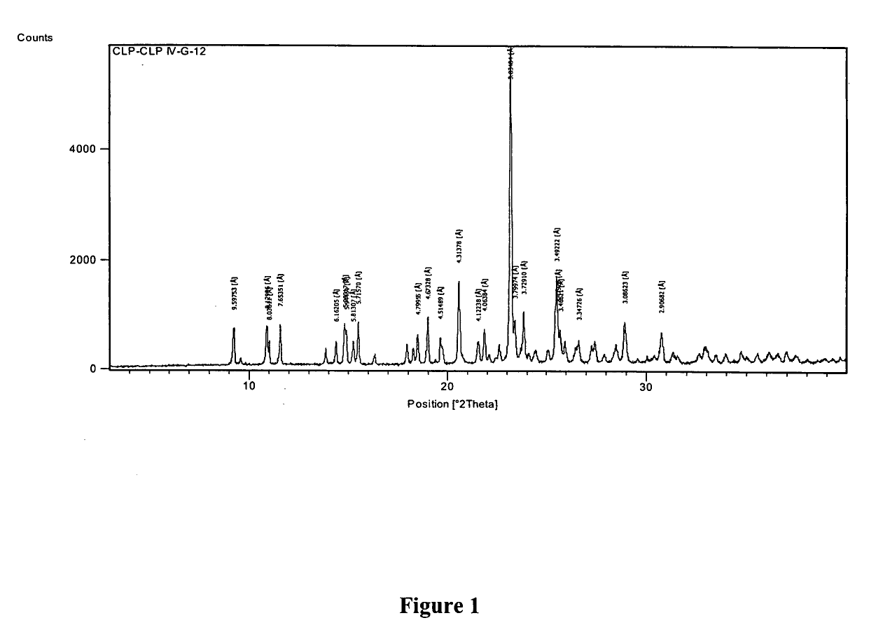

Clopidogrel base (100 g) was dissolved in ethyl acetoacetate (600 ml) at room temperature. This mixture was cooled to −10° C. and concentrated sulphuric acid (98%, density=1.83) was added (15.5 g) while maintaining a temperature of −10° to 0° C. during the acid addition. The reaction mass was stirred for 1.0 hour and warmed slowly to 10 to 15° C. in 30 to 45 minutes. The formed crystals were stirred for another 10 hours. The reaction mass temperature was further raised to 28 to 30° C. and maintained for 2 hours. The solid obtained was filtered under suction and washed with acetone, and dried in an oven at 48° C. for 3 hours. The solid after drying weighed 96 g and was Form I clopidogrel hydrogen sulphate (the PXRD pattern is identical with FIG. 1).

### EXAMPLE 2

Clopidogrel base (100 g) was dissolved in 4-chloro-ethyl acetoacetate (600 ml) at room temperature. This mixture was cooled to −10° C. and concentrated sulphuric acid (98%, density=1.83) was added (15.5 g) while maintaining a temperature of −10′ to 0° C. during the acid addition. The reaction mass was stirred for 1.0 hour and warmed slowly to 10 to 15° C. in 30 to 45 minutes. The formed crystals were stirred for another 10 hours. The reaction mass temperature was further raised to 28 to 30° C. and maintained for 2 hours. The solid obtained was filtered under suction and washed with acetone, and dried in an oven at 48° C. for 3 hours. The solid after drying weighed 95 g and was Form I clopidogrel hydrogen sulphate (the PXRD pattern is identical with FIG. 1).

### EXAMPLE 3

Clopidogrel base (100 g) was dissolved in a mixture of ethyl acetoacetate (500 ml) and acetone (100 ml) at room temperature. This mixture was cooled to −10° C. and concentrated sulphuric acid (98%, density=1.83) was added (15.5 g) while maintaining a temperature of −10° to 0° C. during the acid addition. The reaction mass was stirred for 1.0 hour and warmed slowly to 10 to 15° C. in 30 to 45 minutes. The formed crystals were stirred for another 10 hours. The reaction mass temperature was further raised to 28 to 30° C. and maintained for 2 hours. The solid obtained was filtered under suction and washed with acetone, and dried in an oven at 48° C. for 3 hours. The solid after drying weighed 97 g and was Form I clopidogrel hydrogen sulphate (the PXRD pattern is identical with FIG. 1).

### EXAMPLE 4

Clopidogrel base (5.79 kg) was dissolved in methyl ethyl ketone (37 liter) at room temperature. This mixture was cooled to −15° C. and concentrated sulphuric acid (96%, density=1.83) was added (1.02 liter) while maintaining a temperature of −10° to 0° C. while addition. The reaction mass was stirred for 1.0 hour and warmed slowly to 10 to 15° C. for 30 to 45 minutes. The formed crystals were stirred for another 10 hours. The reaction mass temperature was further raised to 28 to 30° C. and maintained for 15 hours. The solid obtained was filtered under suction and washed with methyl ethyl ketone, and dried in an oven at 48° C. for 3 hours. The solid after drying weighed 4.5 kg and was Form I clopidogrel hydrogen sulphate (the PXRD pattern is identical with FIG. 1).

### EXAMPLE 5

Clopidogrel base (5.79 kg) was dissolved in cyclopentylmethyl ether (37 liter) at room temperature. This mixture was cooled to −10° C. and concentrated sulphuric acid (96%, density=1.83) was added (1.02 liter) while maintaining a temperature of −10 to 0° C. during the acid addition. The reaction mass was stirred for 1.0 hour and warmed slowly to 10 to 15° C. in 30 to 45 minutes. The formed crystals were stirred for another 7 hour. The reaction mass temperature was further raised to 28 to 30° C. and maintained for 8.0 hours. The solid obtained was filtered under suction and washed with cylcopentylmethylether, and dried in an oven at 48° C. for 3 hours. The solid after drying weighed 6.82 kg (90%) and was Form I clopidogrel hydrogen sulphate (the PXRD pattern is identical with FIG. 1).

### EXAMPLE 6

Clopidogrel base (5.79 g) was dissolved in diisopropylglycol (60 ml) and acetone (10 ml) at room temperature. This mixture was cooled to −10° C. and concentrated sulphuric acid (96%, density=1.83) (1.02 ml) was added while maintaining a temperature of −10° to −5° C. during the acid addition. The reaction mass was stirred for 2.0 hour and warmed slowly to 10 to 15° C. in 30 to 45 minutes. The formed crystals were stirred for another 7 hours. The reaction mass temperature was further raised to 28 to 30° C. and maintained for 8.0 hours. The solid obtained was filtered under suction and washed with cylcopentylmethylether, and dried in an oven at 48° C. for 3 hours. The solid after drying weighed 5.2 g and was Form I clopidogrel hydrogen sulphate (the PXRD pattern is identical with FIG. 1).

### EXAMPLE 7

Clopidogrel base (5.79 g) was dissolved in methylpropylether (40 ml) and 10 ml methylpropylketone at room temperature. This mixture was cooled to −15° C. and concentrated sulphuric acid (1.02 ml) (96%, density=1.83) was added while maintaining a temperature of −15 to −10° C. during the acid addition. The reaction mass was stirred for 1.0 hour and warmed slowly to 10 to 15° C. in 30 to 45 minutes. The formed crystals were stirred for another 4 hour. The reaction mass temperature was further raised to 28 to 30° C. and maintained for 10 hours. The solid obtained was filtered under suction and washed with methylpropylketone, and dried in an oven at 48° C. for 3 hours. The solid after drying weighed 6.5 g and was Form I clopidogrel hydrogen sulphate (the PXRD pattern is identical with FIG. 1).

### EXAMPLE 8

Clopidogrel base (5.79 g) was dissolved in t-butyl methyl ether (35 ml) and acetone (10 ml) at room temperature. This mixture was cooled to −15° C. and concentrated sulphuric acid (1.02 L) (96%, density=1.83) was added while maintaining a temperature of −10° to 0° C. during the acid addition. The reaction mass was stirred for 1.0 hour and warmed slowly to 10 to 15° C. in 1 hour. The formed crystals were stirred for another 5 hours. The reaction mass temperature was further raised to 28 to 30° C. and maintained for 3.0 hours. The solid obtained was filtered under suction and washed with t-butyl methyl ether, and dried in an oven at 48° C. for 3 hours. The solid after drying weighed 6.7 kg (90%) and was Form I clopidogrel hydrogen sulphate (the PXRD pattern is identical with FIG. 1).

### EXAMPLE 9

Clopidogrel base (5.5 g) was dissolved in propylmethyl cellosolve (40 ml) at room temperature. This mixture was cooled to −10° C. and concentrated sulphuric acid (1.1 ml) (90%) was added while maintaining a temperature of −10° to 0° C. during the acid addition. The reaction mass was stirred for 1.0 hour and warmed slowly to 15 to 20° C. in 2 hours. The formed crystals were stirred for another 3 hours. The reaction mass temperature was further raised to 28 to 30° C. and maintained for 8.0 hours. The solid obtained was filtered under suction and washed with acetone, and dried in an oven at 48° C. for 3 hours. The solid after drying weighed 5.9 g and was Form I clopidogrel hydrogen sulphate (the PXRD pattern is identical with FIG. 1).

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

We claim:

1. A process for preparation of Form I crystals of (+)-(S)-clopidogrel hydrogen sulphate of Formula IB 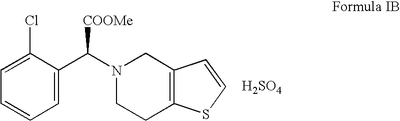
comprising the step of forming said clopidogrel hydrogen sulphate Form I from either
(a) a Type I organic solvent possessing two or more functional groups where at least one functional group is different from the other(s); or
(b) a Type-II solvent selected from the group consisting of methyl ethyl ketone, cyclopentylmethyl ether, dipropylglycolether, dibutylglycol ether, propylmethyl cellosolve, butylmethylcellosolve, propylethylellosolve, butylethylcellosolve, and combinations thereof.

2. The process as claimed in claim 1, wherein the functional groups of the Type-I solvent are selected from the group consisting of ester, ketone, hydroxyl, nitro, nitrile, amides, ethers, and halogen.

3. The process as claimed in claim 1, wherein the functional group combinations in the solvent are ester-ketone, ester-nitrile, halogen-ester, hydroxyl-ester, halogen-ethers, or ketone-halogens.

4. The process as claimed in claim 1, wherein the Type I solvent comprises 4 to 12 carbons.

5. The process as claimed in claim 1, wherein the Type-I solvent is ethyl acetoacetate, methyl acetoacetate, 4-chloroethylacetoacetate, 3-nitroethylpropionyl acetate, propyl lactate, or ethyl lactate.

6. The process as claimed in claim 1, wherein the forming step comprises
(i) dissolving methyl (+)-(S)-clopidogrel base or its salt in the Type I or Type II solvent;
(ii) cooling said clopidogrel containing solution;
(iii) allowing said salt mixture to precipitate (+)-(S)-clopidogrel hydrogen sulphate in Form I; and
(iv) recovering said crystals of Form I.

7. The process as claimed in claim 6, wherein the Type I or Type II solvent is ethyl acetoacetate, methyl acetoacetate, 4-chloroethylacetoacetate, 3-nitroethylpropionyl acetate. propyl lactate, and ethyl lactate, cyclopentylmethyl ether, dipropylglycolether, dibutylglycol ether, propylmethyl cellosolve, butylmethylcellosolve, propylethylellosolve, butylethylcellosolve.

8. The process as claimed in claim 1, wherein said Type I or Type II solvent is in a mixture with one or more other solvent selected from the group consisting of ester, C3-C8 aliphatic or alicylcic ketone, C3-C6 chain or branched chain alcohol, and ether.

9. The process as claimed in claims 8, wherein said other solvent is less than 25 weight percent relative to the Type I or Type II solvent.

10. The process as claimed in claim 8, wherein the ester is propylacetate or butyl acetate.

11. The process as claimed in claim 8, wherein the ketone is acetone, methyl ethyl ketone, methylisopropyl ketone, methylpropyl ketone, or methylisobutylketone.

12. The process as claimed in claim 8, wherein the alcohol is butanol or pentanol.

13. The process as claimed in claim 8, wherein the ether is tertiary butyl methyl ether or cyclopentyl methyl ether.

14. The process as claimed in claim 6, further comprising the step of adding sulfuric acid to the cooled solution to form hydrogen sulfate salt.

15. The process as claimed in claims 14, wherein the solution of clopidogrel base in said solvents is cooled to a temperature of below 20° C. before adding sulphuric acid.

16. The process as claimed in claims 14, wherein the addition of said concentrated sulphuric acid is carried out while maintaining reaction solution temperature between −20 to 10° C.

17. The process as claimed in claims 14, wherein the clopidogrel base solution in said solvent is seeded with Form I prior to addition of sulphuric acid.

18. The process as claimed in claims 14, wherein said sulphuric acid containing mixture is maintained at a temperature range of 0° to 30° C.

19. The process as claimed in claim 14, wherein the strength of said sulphuric acid is about 80 to 98%.

20. The process as claimed in claim 14, wherein the molar ratio of the sulphuric acid to the (+)-(S)-clopidogrel base is about 1:1 to about 1.1:1.

21. The process as claimed in claims 14, wherein the sulphuric acid is incorporated as a solution in a carrier solvent.

22. A stable clopidogrel hydrogensulphate Form I substantially free of Form II.

23. A pharmaceutical composition comprising clopidogrel bisulphate Form I prepared according to claim 1 which is incorporated into a pharmaceutically acceptable dosage form.
